(19) United States
(12) Patent Application Publication
(10) Pub. No.: US 2026/0020869 A1
(43) Pub. Date: Jan. 22, 2026

(54) VENOARTERIAL SHOCKWAVE THERAPY

(71) Applicant: DEERFIELD CATALYST, LLC, New York, NY (US)

(72) Inventor: Mark GELFAND, New York, NY (US)

(21) Appl. No.: 19/099,671

(22) PCT Filed: Jul. 27, 2023
(86) PCT No.: PCT/US2023/071107

§ 371 Date: Jan. 29, 2025

Related U.S. Application Data
(60) Provisional application No. 63/393,355, filed on Jul. 29, 2022.

(51) Int. Cl. A61B 17/22; A61B 17/00; A61M 25/10

(52) U.S. Cl. CPC A61B 17/22022; A61B 2017/00292; A61B 2017/00778; A61B 2017/22024; A61B 2017/22025; A61B 2017/22062; A61M 2025/1072

(57) ABSTRACT

A method includes the step of inserting a catheter inside a vein adjacent to an artery and inflating a balloon within the vein. A focus of a reflector for a shockwave emitter on the catheter is adjusted to be within the artery. The method further includes generating a shockwave within the balloon and directing the shockwave via the reflector through the wall of the vein and the wall of the artery.

### FIELD OF INVENTION

The present invention relates to shockwave therapy to treat calcifications and calcified plaque deposits in cardiovascular central and peripheral blood vessels.

### BACKGROUND

Calcification is a hallmark of advanced atherosclerotic disease and is often hard to treat. Stenosis and chronic total occlusions (CTOs) are frequently encountered in patients with both coronary and peripheral artery disease. These occlusions are associated with a higher risk of adverse events, decreased quality of life, worse procedural success and treatment outcomes, and increased healthcare costs. As a result, vessel preparation (crossing CTOs and preparing lesions) has become an important initial stage in revascularization strategies for treating calcified peripheral artery disease. Plaque modification and CTO crossing devices have enhanced the treatment of moderately calcified cardiovascular lesions.

However, highly calcified stenoses and CTOs continue to create challenges for achieving optimal treatment outcomes and pose a management dilemma for physicians due to the technical/procedural complexities and clinical uncertainties. Peripheral artery disease (PAD) has been associated with significant morbidity and mortality. Critical limb ischemia (CLI), the late stage and worst form of peripheral artery disease, is characterized by multilevel and multivessel arterial stenoses, and an increasing risk for limb loss, and death.

Peripheral artery disease affects 200 million people worldwide, including 20 million people in the United States. It is estimated that 25-30 million people worldwide and greater than 3 million in the United States alone are burdened with CLI. Prognosis of a CLI patient is poor: within the first year of CLI diagnosis, 25% of patients die and 25% will have a major limb amputation as primary treatment, at a cost of $22 billion in the US alone. Two years post below-the-knee (BTK) amputation, 15% will undergo above-the-knee (ATK) amputation and 30% will die.

Moderate to severe calcium is present in 50% of peripheral artery disease patients with severe claudication (cramping pain in the leg is induced by exercise), and the numbers are even higher (>65%) in CLI patients. Peripheral chronic total occlusions are encountered in up to 50% of peripheral artery disease and CLI patients undergoing endovascular treatment of femoropopliteal (FP) and tibial or below-the-knee (BTK) arteries. These obstructions compromise the viability of the affected tissues and ultimately threaten limb loss (amputations).

Chronic total occlusions (CTOs) are often seen in patients with PAD and can occur in multiple vessels. CTOs are dense, collagenous, and have varying degrees of calcification, obstructing blood flow to distal vascular beds. Given their obstructive nature, attempts to cross CTOs during catheter-based interventions are often unsuccessful. Unless a CTO can be traversed, no therapy can be delivered and only bypass options are left. It is always preferable to cross the CTO following and restoring the natural lumen.

The traditional way of crossing the CTO centers on obtaining common femoral artery access and traversing the occlusion from a proximal approach toward the foot. Success with this approach is inconsistent, with failure rates of up to 40%. This is because the ends of CTOs can be in the form of heavily calcified plaque with surfaces that are concave or convex. Such surfaces are difficult or impossible to penetrate. Attempts often result in the wire missing its target and/or penetrating the arterial wall.

Tibio-pedal access has been utilized to increase the success rate of crossing a CTO but has its own technical and clinical limitations. For long (>10 cm) and heavily calcified CTOs, retrograde tibio-pedal access tends to be used most often. However, because the calcified plaque caps can have hardened concave or convex surfaces, they can still be difficult to cross. Thus, there remains a need to soften and fracture the calcified plaque caps of CTOs prior to crossing with a wire, followed by balloon dilation and stenting.

Heavily calcified plaque also poses problems for other types of cardiovascular interventions. Similar to CTO crossing, there is a well-recognized need for calcium fragmentation in challenging revascularization procedures such as atherectomy and plaque modification. Other potential applications where calcium fragmentation can be beneficial are placement of aortic stent-grafts, aorto-iliac stent grafts and aortic valves.

### SUMMARY

The inventors conceived of a system and method that applies shockwaves, e.g., shockwave lithotripsy to an artery from an adjacent vein. Veins are elastic and do not contain calcium. For that reason, they are minimally affected by shock waves. Veins are larger than arteries and safer to puncture. Larger caliber devices can be used in the venous system compared to the arterial ones. For example, 12-14F catheters can be used instead of 6-9F allowing for more power, better cooling, and larger volume of acoustic energy coverage. Veins take blood to the lungs where an accidental blood thrombus and dislodged debris may be arrested without damage to an extremity, brain, or heart.

In one aspect of the technology, an acoustic shockwave generating system includes an energy emitter and a targeting system.

In yet another aspect of the technology, a shockwave is generated within a vein and is directed through a wall of the vein and into a region of calcification in an artery adjacent the vein.

The method may be broadly applied to calcification in the walls of a target artery, to the calcified plaque occluding or partially occluding the artery, and relevant heart anatomy, such as valvular annulus of a heart valve. The method may also be applied to assist the dilation of vessels with calcified walls with a balloon by breaking up the calcified plaque to allow dilation. The method may also be applied to the penetration and wire crossing of calcified plaque in completely occluded arteries.

Veins are often arranged in tight parallel couplings to corresponding arteries separated only by some connective tissue. For example, arteries in the leg above and below the knee that often contain calcified plaque, such as the superficial and deep femoral arteries and the popliteal artery, have companion veins.

In another aspect of the technology, an intravascular lithotripsy (IVL) system and therapy is configured to be positioned within in a venous system and target and fracture calcium in an adjacent artery. The therapeutic catheter contains one or several shock wave emitters (e.g., spark gap emitters) spaced along the shaft of the catheter. Energy discharged from the spark gap mounted on the catheter shaft results in the formation of a plasma vapor bubble, which is immediately followed with the acoustic shockwave. The shock wave energy travels in all directions and passes through fluid and the material of a targeting device (except where intentionally reflected). The shockwave also travels through blood and soft tissue without injury or dangerous heating. When the shockwave reaches the targeted artery, which may be 1-10 mm away, the energy is absorbed by the calcium and causes the calcium to fracture.

Shockwaves travel through soft tissue with minimal effect because of similar acoustic impedance characteristics of water and soft tissue (i.e., water, 1.5×10⁶ kg/m²s; muscle, 1.7×10⁶ kg/m²s). When shockwaves encounter tissue with differing acoustic impedances, such as the transition from soft tissue to calcified tissue (acoustic impedance of calcified tissue is about 7.8×10⁶ kg/m²s. The mechanism by which shockwaves can fracture calcified tissue is attributed to stresses created by the peak positive and negative pressures generated during the shockwave by multiple mechanisms, including compressive, spalling, super-focusing, shearing, and squeezing stresses.

The targeting device may be in the form of a collapsible and expandable balloon and may serve several purposes. Balloon material (e.g., medical grade polymer) may be acoustically matched to the surrounding soft tissue and, specifically, venous wall tissue to minimize the deposition of energy in the polymer wall, which could lead to the rupturing or heating of the balloon. The balloon may be inflated to a relatively low pressure sufficient to distend the vein and ensure apposition to the walls, if desired. Cooling fluid may be pumped in and out of the balloon to cool the spark emitter between the shocks. The balloon may be cycled between the inflated and deflated states to facilitate cooling. Cooling fluid may be chilled outside of the body to just above freezing point to further speed up cooling of the catheter and adjacent tissues. The balloon may be made of a dielectric polymer to prevent electric shock to the patient. Temperature sensors may be integrated in the design of the catheter to monitor temperature inside the balloon, on the catheter shaft or on the balloon surface.

The catheter may be connected to a controller console that contains power electronics, a microprocessor with software, and a user interface needed to generate high voltage electric pulses that generate the spark that generates the acoustic shock waves. The software embedded in the console may execute an automated firing sequence to fracture calcium by automated application of multiple shocks spaced in time while maintaining the temperature in the acceptable range to protect the catheter and the patient from burns.

The targeting device may be placed in a vessel adjacent to the target artery; and energy may be directed and focused, while maintaining safe pressure levels of local pressure and preventing excessive soft tissue damage.

To direct energy towards the target artery and/or focus it on the arterial lumen during treatment, high-energy shockwaves may be condensed or focused through a process of semi-parabolic (or other convex shape) acoustic mirror reflection and combined with other catadioptric refraction and reflection acoustic lenses (all called acoustic shock wave focusing for simplicity) to concentrate their fragmenting impact on the calcium deposits.

Energy discharged from the emitter (e.g., spark gap electrodes) results in the formation of a plasma vapor bubble, which immediately follows the initial acoustic shockwave that can be reflected from the section of the wall of the balloon that is intentionally made reflective. For example, a double balloon with gas trapped between layers can serve as an almost ideal ultrasound mirror. Alternatively, a metallic coating can be deposited on the balloon surface with sufficient cooling. It is appreciated that the metallic coating may only partially reflect acoustic energy but that may be sufficient since perfect focus (or focal point concentration of energy) is not required to achieve the desired pressure at the target range and may even be undesirable for safety reasons. During the therapy procedure the balloon may be rotated with the acoustic aperture (the section of the balloon that does not reflect acoustic waves) facing the target artery and the reflector mirror backing facing the opposing wall of the vein.

The catheter can include a targeting device, such as a semi-compliant inflatable balloon, and may integrate one, two or more radiopaque lithotripsy emitters that can be spaced 2-10 mm apart along the shaft of the catheter as well as conventional radiopaque markers at the proximal and distal edges of the balloon. The emitters receive electrical pulses from the generator vaporizing the fluid (e.g., a traditional mixture of 50% NaCl 0.9% saline and 50% radiopaque contrast can be used) within the balloon and create a rapidly expanding and collapsing bubble. This bubble can transmit unfocused circumferential pulsatile mechanical energy into the vessel wall, in the form of sonic pressure waves equivalent to approximately 50 atmospheres (atm) at the target. If an acoustic mirror is integrated in the balloon design, energy can be somewhat focused on the distance where the calcified artery can be expected, based on the general anatomy of the treated location or the angiographic measurement using digital imaging, computer tomography (CT) or real time ultrasound imaging. The balloons may be made in diameters ranging from 5 mm to 25.0 mm with a standard length of 20 to 200 mm or whatever is required for the targeted vein artery combination.

The intravascular lithotripsy therapy cycle can consist of a series of 1-50 pulses (1 cycles can be applied) or more at 1 pulse per second or at a maximum rate allowed by cooling to maintain safe temperature. The number of therapies (shocks and shock series or pulse trains) needed per lesion will depend on lesion size, vessel diameter and density of calcification.

In one aspect of the technology an acoustic shockwave generating device is configured to be inserted into a vein adjacent to an artery. The acoustic shockwave generating device includes a catheter with an energy emitter configured to generate a shockwave. A targeting device is configured to direct the shockwave through a wall of the vein and through a wall of the adjacent artery. The targeting device is configured to focus the shockwave on a calcium deposit in the artery with enough energy to fragment the calcium deposit.

The targeting device may be in the form of an inflatable balloon. The energy emitter may be positioned inside the targeting device. The targeting device may include an internal membrane that forms a first chamber of the targeting device and a second chamber of the targeting device. The catheter may include at least one infusion port that is configured to deliver a mixture of saline and radiocontrast to the first chamber and at least one infusion port to deliver a gas to the second chamber to inflate the balloon.

The membrane may have a reflecting surface configured to reflect and focus the shockwave generated by the energy emitter on the calcium deposit in the artery. The membrane may have a concave surface that faces the energy emitter. The curvature of the membrane may be adjustable. The first chamber may be configured to be inflated at a higher pressure than the second chamber to adjust the shape of the membrane. The targeting device may be configured so that the curvature of the membrane and a location of a zone of concentrated energy is adjusted by manipulating a ratio of the pressure in the first chamber to the pressure in the second chamber.

The membrane and the zone of concentrated energy may be on opposite sides of the central longitudinal axis of the catheter. The targeting device may be configured to be inserted into the vein in a collapsed state and may be configured to be expanded at a target location within the vein. The catheter may include a central longitudinal axis, and the membrane is configured to reflect, concentrate and direct the shockwave in a direction that is radially oriented relative to the central longitudinal axis of the catheter. The energy emitter may be in the form of one or more pairs of spark gap electrodes.

Another aspect of the technology may be an acoustic shockwave generating system that includes the acoustic shockwave generating device disclosed above and an energy delivery console connected to the catheter and configured to transmit energy to the catheter. The energy delivery console includes an integrated programmable controller. The energy delivery console may include a device configured to deliver inflation and cooling liquid to the targeting device. The energy delivery console may include a device configured to deliver gas to the targeting device for the reflector.

Another aspect of the technology includes a method for fracturing a calcified occlusion from a vein adjacent to an artery containing the calcified occlusion. The method includes inserting a catheter with an energy emitter into the vein, actuating the energy emitter to generate a shockwave within the vein, and directing the shockwave through a wall of the vein and through a wall of the artery to deposit energy in the artery and fragment the calcified occlusion.

The catheter may include an adjustable reflector and the reflector reflects and focuses the shockwave on the calcified occlusion. The method may also include adjusting a curvature of the reflector to manipulate an intensity and a location of a zone of concentrated energy of the shockwave. The reflector may be positioned within a fluid container and divides the fluid container into a first chamber and a second chamber. The shape of the reflector may be manipulated by adjusting a pressure difference between the first chamber and the second chamber.

Another aspect of the technology includes a method that includes inserting a catheter with a shockwave generator inside a vein adjacent to an artery, actuating the shockwave generator to emit a shockwave from within the vein, directing the shockwave through a wall of the vein and through a wall of the artery, and focusing the shockwave on a calcified occlusion in the artery.

The shockwave may be directed in a radial direction. The method may also include providing a first fluid and a second fluid to the catheter and focusing the shockwave by adjusting a pressure differential between the first and second fluids. The first fluid may be a liquid. The second fluid may be a gas. The method may further include focusing the shockwave outside of the vein and inside the artery.

Another aspect of the technology includes a method of treating calcified plaque in an artery. The method includes advancing a catheter through a vein and positioning a shockwave emitting portion of the catheter in the vein adjacent a calcified region of an artery, generating a shockwave within the vein by a shockwave emitter at the shockwave emitting portion of the catheter, directing at least a portion of the shockwave towards the calcified region of the artery, and breaking at least a portion of the calcified plaque in the calcified region of the artery.

Energy from a portion of the shockwave reaching the artery may fracture calcium deposits in the artery. The shockwave emitter may be located at a distal region of the catheter. The method may further include adjusting the shockwave emitter so that a focus of the shockwave is located at the calcified plaque. The focus may be located at a cap of the calcified plaque. The shockwave emitter may include an inner compartment filled with a first fluid and an outer compartment filled with a second fluid, and wherein the compartments are separated by a reflective membrane.

The method may further include adjusting a shape of the reflective membrane to adjust a position of the focus. The shape of the reflective membrane may be manipulated by adjusting a pressure differential between the inner compartment and the outer compartment. The shockwave may be directed in a direction that is radially oriented relative to the central longitudinal axis of the catheter.

Another aspect of the technology includes a method that includes advancing a distal region of a catheter through a vein to a region of the vein proximate a calcified region of an artery, generating a shockwave within the vein using the distal region, directing at least a portion of the shockwave from the vein towards the calcified region of the artery, and breaking at least a portion of the calcified region with the shockwave.

The method may further include identifying the calcified region by imaging the artery in conjunction with the advancing the distal region of the catheter. The method may further include imaging the artery and the vein during the advancing of the distal region. The distal region may include a reflector and the method further comprises moving the reflector to orient the reflector to reflect the shockwave towards the calcified region of the artery. The moving of the reflector may include turning the reflector about a longitudinal axis of the distal region.

The method may further include adjusting a shape of the reflector to align a focus of the reflector at the calcified region of the artery. The method may further include after the breaking of at least the portion of the calcified region, attempting to advance a second catheter into the calcified region to further break the calcified region.

The method may further include, in response to resistance to the advancement of the second catheter into the calcified region, generating a second shockwave using the distal region, directing at least a portion of the second shockwave towards the calcified region of the artery, breaking at least a portion of the calcified region with the second shockwave, and after the second shockwave, attempting to again to advance the second catheter into the calcified region.

The method may further include attempting to advance a second catheter into the calcified region and determining that the second catheter will not safely advance into the calcified region, before the advancement of the distal region of the catheter through the vein.

Another aspect of the technology includes a shockwave generating system that includes a catheter with a distal region configured to be inserted into a vein at a location that is adjacent to a calcified region of an artery. The distal region of the catheter is configured to generate a shockwave within the vein and direct the shockwave from the vein through a wall of the vein and through a wall of the artery towards the calcified region of the artery. The shockwave is intense enough to fracture the calcium deposits in the calcified region.

The distal region of the catheter may be configured to be advanced into the vein in conjunction with the identification of the calcified region by imaging the artery. The distal region may include a reflector and the reflector may be configured to be oriented to reflect the shockwave towards the calcified region of the artery. The shape of the reflector may be adjustable to align a focus of the shockwave at the calcified region of the artery.

Another aspect of the technology includes an acoustic shock wave generating device configured to be inserted into a vein adjacent to an artery. The acoustic shockwave generating device includes a catheter with an energy emitter configured to generate a shockwave and a targeting device configured to direct the shockwave through a wall of the vein and through a wall of the adjacent artery. An energy delivery console is connected to the catheter configured to transmit energy to the catheter. The energy delivery console includes an integrated programmable controller. The targeting device is configured to focus the shockwave on a calcium deposit in the artery with enough energy to fragment the calcium deposit.

The targeting device may include a collapsible acoustic mirror. The targeting device may have multiple compartments separated by the collapsible acoustic mirror, and wherein the acoustic mirror is in the form of a flexible membrane. The acoustic mirror may be configured so that the shape of the acoustic mirror is controlled by regulating a pressure differential between the compartments. The acoustic mirror may be configured so that a shape of the acoustic mirror is manipulated by an amount of fluid delivered to the targeting device.

The energy emitter may be configured to receive electrical pulses from the energy delivery console with enough energy to vaporize the fluid. The acoustic shockwave generating device may be configured to generate and focus a shockwave at a target focal volume. A location and intensity of the focal volume may be based on the shape of the acoustic mirror. The acoustic shockwave generating device may be configured to generate and focus a shockwave so that the intensity of the shockwave at the focal volume is enough to fracture a calcium deposit.

The acoustic shockwave generating device may be configured to generate and focus a shockwave so that the shockwave has a pressure of 50 atmospheres at a target focal volume. The energy delivery console may include a device configured to deliver inflation and cooling liquid to the targeting device. The energy delivery console may include a device configured to monitor a temperature of and control an inflation and cooling liquid.

The energy delivery console may include a device configured to monitor and control a shockwave generation sequence of shock pulses to the energy emitter based on a temperature of the inflation and cooling liquid. The energy delivery console may include a device configured to deliver gas to the targeting device. The energy delivery console may include a device configured to deliver liquid to the targeting device based on a pressure of gas in a gas compartment of the targeting device.

### SUMMARY OF THE DRAWINGS

FIG. 1 shows an occluded artery and adjacent vein with equipment used for vein to artery therapy.

FIG. 2 is a flow chart illustrating a method for breaking up a deposit of calcified plaque within an artery.

FIG. 3 shows another view of the system shown in FIG. 1.

FIG. 4 is a perspective view of an artery and vein pair with a catheter in the vein.

FIG. 5 shows a perspective view of another artery and vein pair with a catheter in the vein.

FIG. 6 is a cross-sectional view of the artery and vein pair of FIG. 1 with the vein to artery therapy equipment inserted into the vein.

FIGS. 7A and 7B show cross-sectional views of a balloon on the catheter in different inflated states.

### DETAILED DESCRIPTION

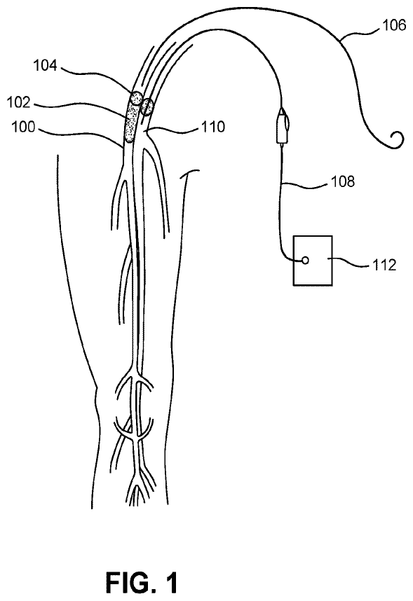

FIG. 1 shows a patient with a femoral artery 100 that has a calcified occlusion 102 with a convex cap 104. A wire or catheter delivery system 106 has been inserted into the femoral artery 100 but cannot penetrate the cap 104. It is contemplated that the catheter delivery system 106 may include a guidewire, a sheath, and a balloon. In addition, a shockwave delivery system 108 has been inserted into an adjacent vein 110. The shockwave delivery system 108 may include a guidewire and, optionally, a sheath. It is contemplated that the shockwave delivery system 108 may also include markers (e.g., echogenic markers) to help with guidance through the vein 110. In addition, the shockwave delivery system 108 may be connected to a console or user interface 112.

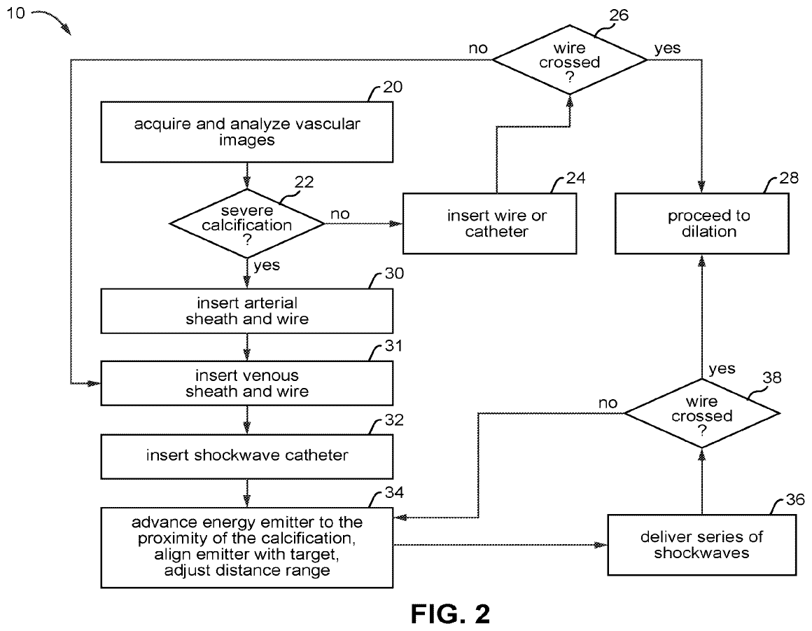

FIG. 2 illustrates an exemplary method or procedure 10 for softening or breaking up calcified plaque within an artery. Regardless of the exact physical location of the targeted calcification, the procedure 10 may start by acquiring and analyzing a vascular image (Step 20) to identify a targeted calcified arterial disease site. Such a site can be in a peripheral, carotid, or cardiac artery.

The identification of calcified plaque and a site for vascular approach may involve the analysis of an arterial angiogram and a venogram performed using X-ray fluoroscopy enhanced by radiocontrast injections (arteriogram and venogram). In addition, ultrasound imaging can be instrumental for identifying instances of calcification in relatively superficial arteries. It is contemplated that magnetic resonance imaging (MRI), cat scan devices, or any other type of imaging equipment capable of generating an image of an internal structure of a patient's body may be used to image and analyze a targeted site.

Once the target site is imaged, the image may be analyzed to determine the severity of the calcification (Step 22), which can then be used to determine which solution will be used to remedy the calcified obstruction. Less severe calcification may be resolved by directly treating the occlusion in the artery without first accessing an adjacent vein. For more severe calcification, the adjacent vein may be accessed, and the calcification treated prior to crossing and distending the stenosis in the targeted artery.

The calcification may be determined to be less severe if there is an accessible pathway within the calcification (i.e., the occlusion is not complete) or if the calcification is soft enough to allow penetration by a wire. The calcification may be determined to be more severe if the occlusion is complete. The calcification may also be determined to be more severe if the calcification has a cap with a concave or convex surface. A less severe calcification may allow for antegrade penetration and crossing of the cap with a wire. More severe calcification may prevent antegrade penetration of the cap with a wire.

If the calcification is determined to be less severe, a sheath and a wire may be inserted into the artery without also inserting a sheath and a wire into the adjacent vein (Step 24). If the wire is able to penetrate the cap or is otherwise able to cross the calcified portion of the artery (Step 26), a dilating balloon may be inflated to break up the occlusion and open up the pathway within the artery (Step 28).

If the calcification is determined to be severe or if the wire or catheter (the catheter delivery system 106) is unable to penetrate the occlusion (Step 30), the shockwave delivery system 108 may be inserted into the adjacent vein (Step 31).

Venous vascular access (e.g., femoral) is generally safe and easy to obtain, and an appropriate size introducer sheath can be placed in advance or during the procedure. Vascular access to the artery 100 and the vein 110 may also be obtained using standard techniques under fluoroscopic guidance.

Once the shockwave delivery system 108 is inserted into the vein 110, a shockwave catheter 114 may be inserted into the vein 110 over the guidewire of the shockwave delivery system 108 (Step 32) and a portion of the shockwave catheter 114 containing an energy emitter 116 and the targeting device 118 may be aligned with the occlusion 102 in the artery 100 (Step 34). The energy emitter 116 and the targeting device 118 may be located in a distal region of the shockwave catheter 114 (i.e., a region on the shockwave catheter 114 that is distal to the user interface 112).

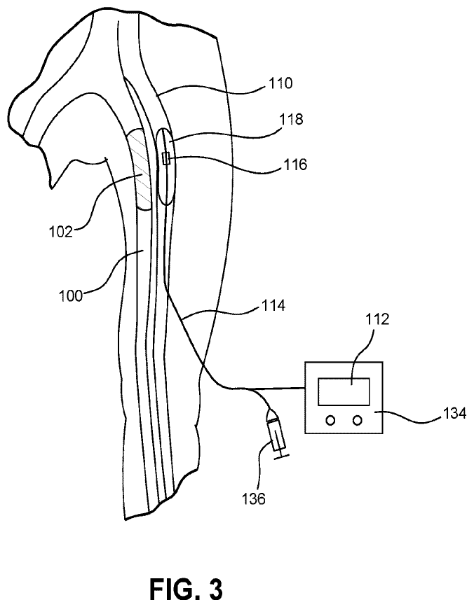

FIG. 3 illustrates an occluded artery 100 and an adjacent vein 110 with a shockwave catheter 114. The shockwave catheter 114 includes an energy emitter 116 that is aligned with the occlusion 102. The energy emitter can be aligned with a proximal cap of the occlusion 102 with the intention of targeting the calcified cap to soften the calcified cap before crossing. The artery 100 can be, for example, an iliac or a femoral or other calcified artery in the patient. The shockwave catheter 114 may be inserted into the adjacent vein 110 and the energy emitter 116 may be aligned with the occlusion 102.

Fluoroscopy, sonography, and other imaging techniques can be used to achieve the alignment. In some embodiments an ultrasound guided intravascular lithotripsy catheter comprises at least one targeting ultrasound transducer, which can be a diagnostic ultrasound transducer, wherein the transducer is positioned on the shockwave catheter 114 in such a way that when the targeting ultrasound transducer is aligned with a vasculature landmark, the shock wave beam is aligned with the target vascular landmark (e.g., the calcification 102). The calcified occlusion 102 may be reflective of ultrasound typically used in intravenous ultrasound type catheters. The vascular landmark can also be an echogenic guidewire or echogenic contrast agent.

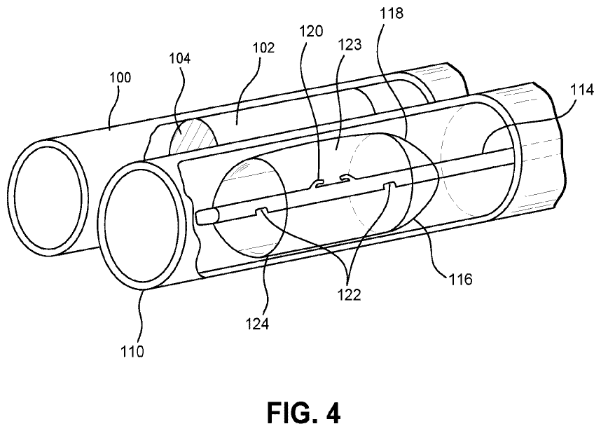

FIG. 4 shows a detailed view of the energy emitter 116 being aligned with the occlusion 102 in the artery 100. The artery 100 is diseased with calcified plaque and may have a total occlusion 102 that is hard to cross with a catheter delivery system 106. The goal of the therapy is to fragment the calcium so that the catheter delivery system 106 can cross the occlusion 102 safely. The shockwave catheter 114 is advanced into the adjacent vein 110 so that the energy emitter 116 and the targeting device 118 are adjacent to the occlusion 102 in the artery 100 and so that a shockwave can be directed at the occlusion 102 (e.g., the convex or concave cap of the occlusion 102).

As can be seen, the energy emitter 116 may be positioned inside the targeting device 118. It is contemplated that the energy emitter 116 may have any suitable form capable of generating a shockwave at the occlusion 102 in the artery 100 from the adjacent vein 110. In some configurations, the catheter delivery system 106 may include one or more pairs of electrodes, e.g., spark gap electrodes.

The targeting device 118 may be collapsible and expandable so that it can be inserted by way of the catheter 114 and then be deployed in the in vein 110. Once positioned in the desired location, the targeting device 118 may be deployed and expanded to distend the vein 110 and ensure apposition to the walls of the vein 110 so that the targeting device 118 (and the rest of the shockwave delivery system 108) is fixed in place. The expansion may be actuated by mechanical, electrical, or pneumatic means. For example, the targeting device 118 may be in the form of an inflatable balloon.

When actuated, the energy emitter 116 may emit soundwaves in all directions including directions that are away from the artery 100. The targeting device 118 may include a reflector 124 that redirects those wayward soundwaves back toward the artery 100. In addition, the reflector 124 may not only be designed to redirect the soundwaves but may also cause the soundwaves to converge at a predetermined distance from the energy emitter 116 inside the artery 100 at the occlusion 102 (the concentrated energy zone). In other words, the reflector 124 may redirect and focus the soundwaves on the occlusion 102 so that the energy emitted by the energy emitter 116 is concentrated on the occlusion 102 and not at a region that is further than or closer than the occlusion 102.

When performing the alignment and focusing Step 34, the targeting device 118 and the energy emitter 116 may be inserted into the vein 110 so that the energy emitter 116 and the targeting device 118 are aligned to face the occlusion 102 and/or the cap 104. Once in position, the targeting device 118 may be inflated with a mixture of saline and radiocontrast liquid. The mixture of saline and radiocontrast liquid may be infused into the targeting device 118 through one or more infusion ports 122. In addition, the targeting device 118 may be made of compliant or non-compliant material capable of extending to the walls of the vein 110 and stabilizing the system.

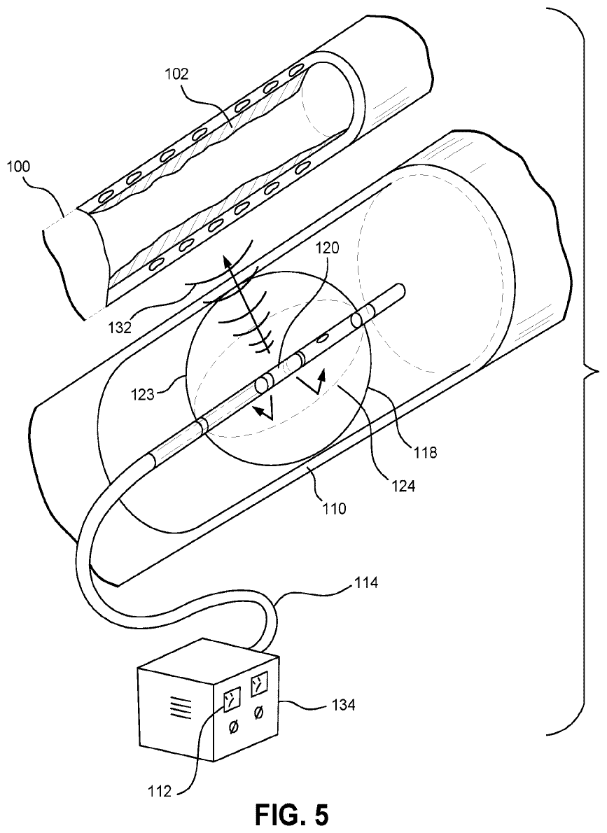

It is contemplated that the targeting device 118 and the energy emitter 116 may be positionally fixed relative to each other on the shockwave catheter 114 so that moving the shockwave catheter 114 moves both the targeting device 118 and the energy emitter 116 in the same way. Alternatively, the targeting device 118 and the energy emitter 116 may be independently movable relative to each other. FIG. 5 shows the energy emitter 116 positioned and aligned relative to the occlusion 102 and emitting a shockwave at the occlusion 102.

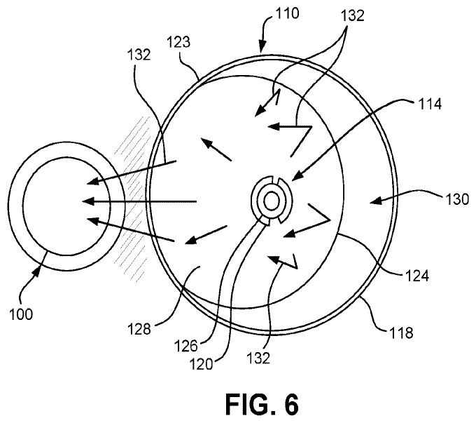

FIG. 6 shows one way to direct the shock waves from the vein 110 to an adjacent artery 100 that is diseased with calcified plaque 102 and may be partially or completely occluded. The catheter 114 is equipped with the energy emitter 116 and the targeting device 18 and may be inserted into the vein 110. It is contemplated that the energy emitter 116 may be laser cut out of a metal tube, which may be a stainless-steel tube. In addition, the energy emitter 116 may include two or more electrodes spaced along the distal portion of the catheter 114.

The targeting device 118 may have multiple compartments separated by the reflector 124, which may be in the form of a flexible membrane. For example, the reflector 124 may an internal component of the targeting device 118 and may divide the targeting device 118 into an inner compartment (or first chamber) 128 and an outer compartment (or second chamber) 130. The energy emitter 116 may be located within the inner compartment 128. In addition, the compartments 128, 130 of the targeting device 118 may be bonded to the shaft of the catheter 114 and bonded together at the boundary of a shockwave directing aperture segment 123 so that they can be inflated and deflated separately by gas (such as, for example, medical grade CO₂) or liquid. In addition, the shockwave catheter 114 may incorporate a guidewire lumen 126 and other lumens for delivery of liquid and gas to the different compartments of the targeting device 118.

Gas may be supplied to the outer compartment 130 of the targeting device 118 and may be trapped between the reflector 124 and the outer surface of the targeting device 118. The gas can be air, nitrogen, CO₂, or other inert gas. It is contemplated that the gas may be soluble in blood to reduce the risk to a patient if the catheter leaks. The gas filled space and/or the reflector 124 may be relatively impermeable to sound or shock waves so that the reflector 124, which forms the boundary between the inner compartment 128 and the outer compartment 130, becomes a reflective surface that reflects the sound or shock waves.

In addition, the inner compartment 128 may be filled with a fluid (liquid or gas) that is more permeable to sound or shock waves. For example, the inner compartment 128 may be filled with water or saline. Because the reflector 124 is flexible, the shape of the reflector 124 may be manipulated to adjust the location and intensity of the zone of concentrated energy. The zone of concentrated energy may be a point or a region at which the shockwave is focused with enough intensity to fracture a calcium deposit (a focal volume). One way to manipulate the shape of the reflector 124 is to regulate a pressure differential between the inner compartment 128 and the outer compartment 130.

For example, liquid may be supplied to the inner compartment 128 at a first pressure and the gas may be supplied to the outer compartment 130 at a second pressure that is higher than the first pressure (for example by 3 atm) with carbon dioxide. This will lead to the compression of gas and the formation of a curved reflection surface. The compartments 128, 130 of the targeting device 118 may have a burst pressure of 30 atm and may operate within a pressure range of 1-12 atm. This difference in pressure between the inner and the outer compartments 128, 130 may warp the reflector 124 into a parabolic or semi-parabolic curved shape, thereby forming a concave (e.g., parabolic, semi-parabolic, cylindrical, etc.) reflecting surface.

The radius of curvature of the curved shape may be increased or decreased by adjusting the pressure differential between the inner compartment 128 and the outer compartment 130. For example, lower pressure differentials may lead to the surface of the reflector 124 having greater radii of curvature (i.e., a flatter surface), while greater pressure differentials may lead to the surface of the reflector 124 having smaller radii of curvature (i.e., a surface with a greater curvature). The adjustment of the shape of the reflector 124 may direct and focus the shockwave beam to a predicted distance and region (the concentrated energy zone) which can be matched to the actual distance to the artery 100 and location of the occlusion 102 within the artery 100. The range of potential shapes of the reflector 124 may depend at least in part on inflation pressure.

The shape of the reflector 124 may range from flat (equal pressures or zero pressure differential) to cylindrical or spherical (zero pressure in one of the compartments due to gas being evacuated and compressed into delivery tubes). Thus, adjusting the pressure differential between the inner and outer compartments 128, 130 allows the reflector 124 to transform through other shapes in between the flat and cylindrical or spherical shapes, thereby allowing a concentration of energy at different depths and locations. A spherical double balloon system or an elongated balloon can be envisioned as well as a series of spherical balloons. The physics of the stretched reflector 124 is such that the reflector 124 will strive to attain a perfect spherical shape as the pressure difference increases.

Referring back to FIGS. 3 and 5, the shockwave catheter 114 may be connected to a control and energy delivery console 134 that is outside of the body. The console 134 may be equipped with the user interface 112 and accessory devices 136 that can deliver inflation and cooling liquid for the targeting device 118 and gas for the reflector 124. Automation of the inflation, deflation, pressure monitoring, temperature monitoring and energy delivery can be integrated with a programmable controller that may be the part of the console 134 or reside at least partially in a tablet computer.

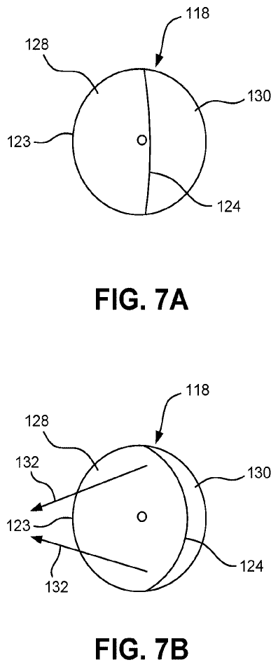

FIGS. 7A and 7B schematically illustrate the targeting device 118 mounted on the shockwave catheter 114 and equipped with the energy emitter 116 being in the form of hydroelectric shock electrodes on the shockwave catheter 114. The targeting device 118 may be partitioned into a liquid section (e.g. saline, water, or other liquid mixture) and a gas section (e.g. CO2 or other inert water soluble gas). Increasing the pressure in the liquid compartment 128 over the pressure in the gas compartment 130 creates a reflecting surface shape with a variable zone of concentrated energy and variable degree of intensity of the soundwaves within the zone of concentrated energy. Such a reflector 124 can be used to direct and focus shock waves in the desired direction for the maximum shock energy deposition in the volume of tissue at a desired distance, such as a calcified area in an artery or in a targeted zone of the heart.

It is contemplated that the reflector 124 and the zone of concentrated energy are positioned on opposite sides of a central longitudinal axis of the shockwave catheter 114. In addition, the entirety of the reflector 124 may be positioned on one side of the central longitudinal axis so that the reflector 124 directs the shockwave in a direction that is radial relative to the central longitudinal axis.

It is contemplated that for different anatomical locations, different geometries and shapes of the reflector 124 may be preferred with the common objective of forming an aperture 123 to direct and at least partially focus or concentrate shock waves emitted by the energy emitter 116 through the aperture 123 and reflect and absorb shockwave energy emitted in the opposite direction.

Referring back to FIG. 2, once the energy emitter 116 and the targeting device 118 are aligned and focused, the energy emitter 116 may be actuated to deliver one or more pulses of energy (Step 36). The number of pulses may be predetermined or may depend on the determination that the cap or occlusion has been softened or broken up.

Once the one or more pulses of energy have been emitted, the catheter delivery system 106 may be reinserted or advanced into the artery 100. If the catheter delivery system 106 is already inside the artery 100, the catheter delivery system 106 may be moved to try to cross the cap 104 of the occlusion 102 (Step 38). If the catheter delivery system 106 successfully crosses the cap 104, Step 28 may be performed. Step 28 may include dilation and stenting of the targeted stenosis to restore blood flow and downstream tissue perfusion. Step 28 may also include traversing the stenosis, and inserting and inflating a balloon inside the artery 100 to break up the remaining portions of the occlusion and open up the pathway within the artery. However, if the catheter delivery system 106 is still unable to penetrate the cap 104 of the occlusion 102, Step 34 may be performed (i.e., realigning, repositioning, and refocusing the energy emitter 116 and the targeting device 118 to a new target location on the occlusion 102).

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise.

### PARTS LIST

10 Method
Step 20
Step 22
Step 24
Step 26
Step 28
Step 30
Step 32
Step 34
Step 36
Step 38
100 Artery
102 Occlusion
104 Cap
106 Catheter delivery system
108 Shockwave delivery system
110 Vein
112 User interface
114 Shockwave catheter
116 Energy emitter
118 Targeting device
122 infusion port
123 Aperture
124 Reflector
126 Lumen
128 Inner compartment
130 Outer compartment
132 Sound waves
134 Console
136 Accessory devices

1.-70. (canceled)

71. A method for fracturing a calcified occlusion from a vein adjacent to an artery containing the calcified occlusion, the method comprising:
inserting a catheter with an energy emitter into the vein;
actuating the energy emitter to generate a shockwave within the vein; and
directing the shockwave through a wall of the vein and through a wall of the artery to deposit energy in the artery and fragment the calcified occlusion.

72. The method of claim 71, wherein the catheter comprises an adjustable reflector and the reflector reflects and focuses the shockwave on the calcified occlusion.

73. The method of claim 72, further comprising adjusting a curvature of the reflector to manipulate an intensity and a location of a zone of concentrated energy of the shockwave.

74. The method of claim 72, wherein the reflector is positioned within a fluid container and divides the fluid container into a first chamber and a second chamber.

75. The method of claim 74, wherein the shape of the reflector is manipulated by adjusting a pressure difference between the first chamber and the second chamber.

76. A method of treating calcified plaque in an artery, the method comprising:
advancing a catheter through a vein and positioning a shockwave emitting portion of the catheter in the vein adjacent a calcified region of an artery, generating a shockwave within the vein by a shockwave emitter at the shockwave emitting portion of the catheter;
directing at least a portion of the shockwave towards the calcified region of the artery, and
breaking at least a portion of the calcified plaque in the calcified region of the artery.

77. The method of claim 76, wherein energy from a portion of the shockwave reaching the artery fractures calcium deposits in the artery.

78. The method of claim 76, wherein the shockwave emitter is located at a distal region of the catheter.

79. The method of claim 76, further comprising adjusting the shockwave emitter so that a focus of the shockwave is located at the calcified plaque.

80. The method of claim 79, wherein the focus is located at a cap of the calcified plaque.

81. The method of claim 76, wherein the shockwave emitter comprises an inner compartment filled with a first fluid and an outer compartment filled with a second fluid, and wherein the compartments are separated by a reflective membrane.

82. The method of claim 81, further comprising adjusting a shape of the reflective membrane to adjust a position of the focus.

83. The method of claim 81, wherein the shape of the reflective membrane is manipulated by adjusting a pressure differential between the inner compartment and the outer compartment.

84. The method of claim 76, wherein the shockwave is directed in a direction that is radially oriented relative to the central longitudinal axis of the catheter.

85. A method comprising:
advancing a distal region of a catheter through a vein to a region of the vein proximate a calcified region of an artery;
generating a shockwave within the vein using the distal region;
directing at least a portion of the shockwave from the vein towards the calcified region of the artery, and
breaking at least a portion of the calcified region with the shockwave.

86. The method of claim 85, further comprising identifying the calcified region by imaging the artery in conjunction with the advancing the distal region of the catheter.

87. The method of claim 85, further comprising imaging the artery and the vein during the advancing of the distal region.

88. The method of claim 85, wherein the distal region includes a reflector and the method further comprises moving the reflector to orient the reflector to reflect the shockwave towards the calcified region of the artery.

89. The method of claim 88, wherein the moving of the reflector includes turning the reflector about a longitudinal axis of the distal region.

90. The method of claim 88, further comprising adjusting a shape of the reflector to align a focus of the reflector at the calcified region of the artery.

91. The method of claim 85, further comprising:
after the breaking of at least the portion of the calcified region, attempting to advance a second catheter into the calcified region to further break the calcified region.

92. The method of claim 91, in response to resistance to the advancement of the second catheter into the calcified region,
generating a second shockwave using the distal region;
directing at least a portion of the second shockwave towards the calcified region of the artery;
breaking at least a portion of the calcified region with the second shockwave; and
after the second shockwave, attempting to again to advance the second catheter into the calcified region.
